(19) United States
(12) Patent Application Publication
(10) Pub. No.: US 2009/0318812 A1
(43) Pub. Date: Dec. 24, 2009

(54) ULTRASOUND WAVEGUIDE

(75) Inventors: Jan Frederik Suijver, Eindhoven (NL); Nijs Cornelis Van Der Vaart, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/515,026

(22) PCT Filed: Nov. 13, 2007
(86) PCT No.: PCT/IB07/54614

§ 371 Date: May 15, 2009

(30) Foreign Application Priority Data
Nov. 21, 2006 (EP) 06124471.1

(51) Int. Cl. A61B 8/00

(52) U.S. Cl. 600/459

(57) ABSTRACT

In a medical therapy or diagnostic system, magnetic resonance (MR) techniques are used in order to determine the position of the focal point of an ultrasound wave with respect to a patient's body. Therefore, it is an object of the present invention to provide an ultrasound waveguide which could be used within a magnetic resonance environment. To better address these concerns, it is an aspect of the present invention to provide an ultrasound waveguide comprising an electrically conducting section and an electrically isolating section. A reduction of the length of an electrically isolating section possibly decreases the electromagnetic coupling of the high frequency electromagnetic field in the MR environment to the electrically conducting section.

### FIELD OF THE INVENTION

The invention relates to the field of ultrasound devices, and more specifically to an ultrasound waveguide.

### BACKGROUND OF THE INVENTION

U.S. Pat. No. 5,443,068 discloses a positioner for a magnetic resonance (MR) surgery system which positions a focal point of an ultrasound transducer to selectively destroy tissue in a region within a patient. The ultrasound transducer is located outside the patient and the ultrasound wave being emitted from the transducer propagates through the patient's skin into the patient's body. Due to the absorption of the ultrasound wave on its path through the patient's tissue the transducer must provide more power than actually required at the focal point in the patient's body.

### SUMMARY OF THE INVENTION

In therapeutic devices combining ultrasound and MR technology it is desirable to use high intensity focused ultrasound (HIFU) waves, being focused at a specifically chosen point in a patient's body. Furthermore there are also further applications of ultrasound technology in therapy, diagnostics and imaging which require the provision of a path for ultrasound waves into a patient in an MR environment. Whereas all aforementioned applications do not necessarily require high intensity ultrasound waves, but could be carried out using lower intensities. Therefore, it is necessary to provide a guidance for the ultrasonic waves to a specific point in a patient's body.

It would be advantageous to provide an ultrasound waveguide which can be used inside a patient's body in an MR environment. It would also be desirable to have an ultrasound waveguide which is flexible and bendable.

To better address one or more of these concerns, in a first aspect of the invention an ultrasound waveguide is provided comprising at least one electrically conducting section with a first acoustical impedance Z₁ and at least one electrically isolating section with a second acoustical impedance Z₂.

Therein the impedance Z of a solid, or more precisely the acoustical field impedance in bulk material, is given by the product of its density ρ and its speed of sound v, i.e. Z=ρv.

MR technology uses high frequency electromagnetic waves in order to determine the spin orientation of molecules in a magnetic field. Ultrasound waveguides are fabricated of metal wires which are coupled to an ultrasound transducer in order to provide a ultrasound waveguide along the longitudinal axis of the wire. However, in the high frequency electromagnetic field of an MR environment electrically conducting materials of a certain extension experience a thermal heat-up due to an electromagnetic coupling of the metal part to the high frequency electromagnetic field, i.e. due to resonance effects and Eddy currents. The heating of a metal part within a patient's body however might cause severe injuries.

The restriction of the electrically conductive part or section of an ultrasound waveguide along at least one dimension by an electrically isolating part or section reduces the coupling of a high frequency electromagnetic field to the electrically conducting waveguide. Since the ultrasound waveguide is formed by a wire whose dimension along its longitudinal axis is much longer than its diameter or thickness in a direction perpendicular to its longitudinal axis, it is desirable to restrict the length of the conductive wire or waveguide section along its longitudinal axis. The electrically conductive section is acoustically coupled to the electrically isolating section in order to provide a transmission of the acoustical wave through the electrically conductive section and further through the electrically isolating section.

Desirably, in an embodiment of the invention there is no reflection loss in intensity or power at the interface between the electrically isolating section and the electrically conducting section. The transmitted fraction of the ultrasound intensity at the interface under perpendicular propagation with respect to said interface is given by

$$\frac{4 {Z}_{1} {Z}_{2}}{{( {Z}_{1} + {Z}_{2} )}^{2}} ,$$

wherein Z₁ and Z₂ are the impedances of the electrically isolating section and the electrically conducting section, respectively. A perfect transmission of the ultrasound wave across the interface between the electrically conducting section and the electrically isolating section is achieved, once the two sections are perfectly impedance matched such that

$$\frac{4 {Z}_{1} {Z}_{2}}{{( {Z}_{1} + {Z}_{2} )}^{2}} = 1 ,$$

i.e. the impedances Z₁ and Z₂ of the electrically conducting section and the electrically isolating section, respectively, are equal. If the impedance matching condition is fulfilled, no internal reflection of the acoustic wave occurs at the interface between the electrically conducting section and the electrically isolating section.

According to a further embodiment of the invention an impedance mismatch leading to a reflection of 10% of the ultrasound intensity coupled into the waveguide is acceptable. However, a reflection of less than 4% would be desirable.

An impedance matching suitable for practical applications can be achieved by using aluminum (Z_{Al}=13400) and fused silica (Z_{fs}=13100) for the electrically conducting section and the electrically isolating section, respectively. In alternative embodiments combinations of indium (Z_{In}=16206) and glass (Z_{gl}=15702.5) or lead (Z_{Pb}=24600) and sapphire (Zₛₐ=25480) could be used for the electrically conducting section and for the electrically isolating section, respectively.

In a further alternative embodiment, a plurality of electrically conducting sections and electrically isolating sections are arranged alternately. With this design a long ultrasound waveguide can be provided, exceeding the critical length of a single electrically conducting waveguide section without an excessive thermal heating.

In a further embodiment of the invention, the length of said conducting section is equal to an integer multiple of the ultrasound wavelength for which the waveguide is made for, plus a quarter of said wavelength. This way, multiple reflections and boundaries between the electrically conducting section and the electrically isolating section can be avoided by destructive interference between reflected and transmitted waves.

Ideally, for a reduction of the heating-up of the electrically conducting section said electrically conducting section has a length of 20 cm or less. In a particular embodiment, the electrically conducting section has a length of 15 cm. This length better addresses the problem of an electromagnetic coupling of the incidental high frequency electromagnetic wave to the ultrasound waveguide.

In a further embodiment, the electrically isolating section has a length in a range from 0.05 mm to 10 mm and in another embodiment the electrically isolating section has a length of 1 mm. This might enhance the flexibility of the ultrasound waveguide provided by the length of conducting material is not disturbed by the inflexible electrically isolating section.

Desirably there is an embodiment of the present invention wherein the electrically conducting section and the electrically isolating section are press-fitted against each other. This way a good transmission of the ultrasound wave along the waveguide via the interfaces between the different sections is provided.

In an embodiment of the present invention the ultrasound waveguide can be employed in a medical instrument. It is desirable to use the waveguide in various types of medical instruments which might be designed for therapy, diagnostics and imaging. Those medical instruments include, but are not restricted to catheters, ultrasound devices for the thermal destruction or ablation of tissue, ultrasound imaging devices or ultrasound endoscopes.

It would be desirable to have the ultrasound waveguide employed in an ultrasound device for the ablation of atrial fibrillation, in which electrically conductive heart tissue is destroyed or ablated by ultrasound in order to restore the normal sine rhythm of a patient's heart. In the described operation the ultrasound waveguide would be guided using MR imaging techniques in order to meet the correct point for the destructive effect of the ultrasound wave.

Concerning ultrasound imaging it would be desirable tom employ the ultrasound waveguide in an ultrasound imaging device which might be used for the imaging of neurovascular structures under MR guidance, such as aneurysms.

These and other aspects of the invention will be apparent from and elucidated with reference to the embodiments described hereinafter.

### BRIEF DESCRIPTION OF EMBODIMENTS

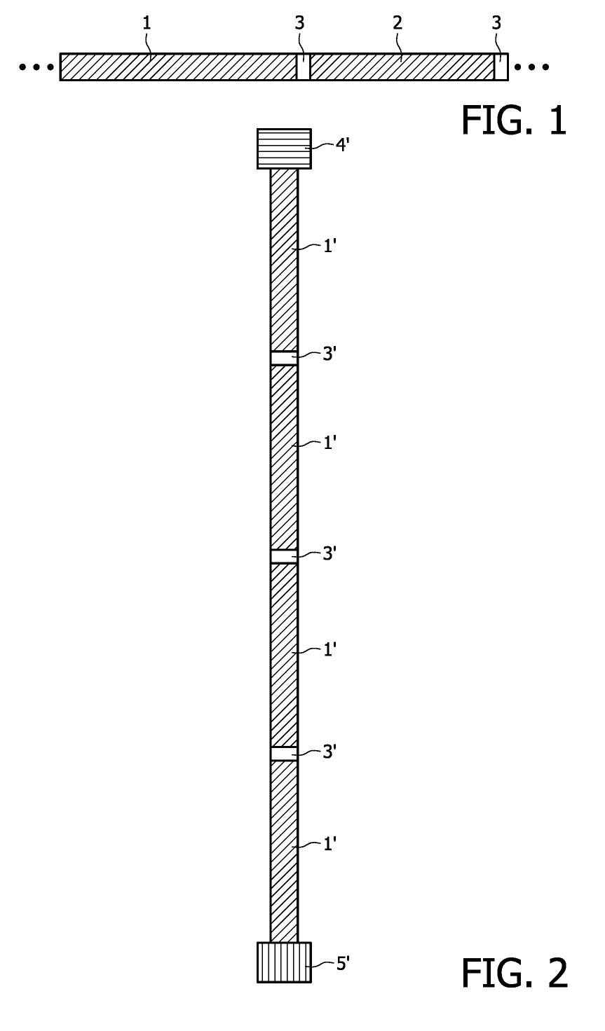

FIG. 1 diagrammatically shows a cutaway view of an ultrasound waveguide according to an embodiment of the present invention.

FIG. 2 shows a diagrammatic view of an ultrasound system using an ultrasound waveguide according to an embodiment of the present invention.

### DETAILED DESCRIPTION OF EMBODIMENTS

In FIG. 1 a cutaway view of an ultrasound waveguide according to a first embodiment of the present invention is diagrammatically shown. The waveguide element shown comprises two conducting sections 1, 2 made of aluminum. While the electrically conducting section 1 has a length of 19 cm, the electrically conducting section 2 has a length of 15 cm. Between the conducting waveguide sections 1, 2 electrically isolating sections 3 are inserted. The electrically isolating sections are made of a 1 mm thick slice of fused silica.

Since aluminum (Z_{Al}=134000) and fused silica (Z_{fs}=131000) are almost perfectly impedance matched there is hardly any reflection at the interfaces between the different sections 1, 2, 3 of the ultrasound waveguide.

As indicated by the bold dots, FIG. 1 exemplary shows a cutaway view. The actual waveguide comprises more than two electrically conducting and electrically isolating sections. As the electrically isolating sections 3 are kept fairly short in length the characteristics of the overall waveguide concerning bending and flexibility are primarily defined by the properties of the electrically conducting aluminum sections 1, 2.

In FIG. 2 an ultrasound system making use of an embodiment of the ultrasound waveguide according to the present invention is diagrammatically shown. An ultrasound wave is coupled into the waveguide from an ultrasound transducer 4′ and at the other end of the waveguide the ultrasound wave is coupled into an ultrasound receiver 5′, in the present case an ultrasound tip for therapeutical purposes. The waveguide in between the transducer 4′ and the receiver 5′ comprises of electrically conductive sections 1′ made of indium and alternately of electrically isolating sections made of glass sections 3′.

The length of the electrically isolating sections 3′ is chosen to be 1 mm in order to provide a thickness which can still be handled without adversely influencing the flexibility of the waveguide as provided by the electrically isolating indium sections 1′. The different sections of the waveguide 1′, 3′ provide a periodical structure. The length of the electrically conducting sections 1′ is chosen such that it corresponds to a whole number of the ultrasound wavelength emitted by the transducer 4′ plus a quarter of this wavelength.

While the invention has been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. The mere fact that certain measures are recited in mutually different dependent claims does not indicate that the combination of these measures can not be used to advantage. Any reference signs in the claims should not be construed as limiting the scope.

1. Ultrasound waveguide comprising
at least one electrically conducting section (1, 1′, 2) with a first acoustical impedance Z₁ and at least one electrically isolating section (3, 3′) with a second acoustical impedance Z₂.

2. Ultrasound waveguide according to claim 1, wherein
$$0.9 \leq \frac{4 {Z}_{1} {Z}_{2}}{{( {Z}_{1} + {Z}_{2} )}^{2}} \leq 1.$$

3. Ultrasound waveguide according to claim 1, wherein a plurality of electrically conducting sections (1, 1′, 2) and electrically isolating sections (3, 3′) are arranged alternately.

4. Ultrasound waveguide according to claim 1, wherein a plurality of electrically conducting sections (1, 1′, 2) and electrically isolating sections (3, 3′) are arranged periodically.

5. Ultrasound waveguide according to claim 1, wherein the length of said conducting section (1, 1′, 2) is equal to an integer multiple of the ultrasound wavelength for which the waveguide is made for plus a quarter of a wavelength.

6. Ultrasound waveguide according to claim 1, wherein said electrically conducting section (1, 1′, 2) has a length of 20 cm or less.

7. Ultrasound waveguide according to claim 1, wherein said electrically isolating section (3, 3′) has a length in a range from 0.05 mm to 10 mm.

8. Ultrasound waveguide according to claim 1, wherein said electrically conducting section (1, 1′, 2) comprises aluminum and the electrically isolating section (3, 3′) comprises fused silica.

9. Ultrasound waveguide according to claim 1, wherein said electrically conducting section (1, 1′, 2) and said electrically isolating section (3, 3′) are press fitted against each other.

10. Medical instrument comprising an ultrasound waveguide according to claim 1.
